(19) United States
(12) Patent Application Publication
(10) Pub. No.: US 2023/0110694 A1
(43) Pub. Date: Apr. 13, 2023

(54) ANIMAL BEDDING AGITATOR

(71) Applicant: ANIMAL CARE SYSTEMS, INC., Centennial, CO (US)

(72) Inventors: Andrew Poulter, Littleton, CO (US); Beverly A. Chua, Denver, CO (US); Robert Tierney, Centennial, CO (US)

(21) Appl. No.: 17/961,301

(22) Filed: Oct. 6, 2022

Related U.S. Application Data
(60) Provisional application No. 63/253,345, filed on Oct. 7, 2021.

(51) Int. Cl. A01K 1/03; A01K 1/035

(52) U.S. Cl. CPC A01K 1/031; A01K 1/035; C12Q 1/6806

(57) ABSTRACT

Devices for agitating animal bedding are disclosed. The devices include a tumbler, a closure mechanism, a rotating mechanism, a motor, and a controller. The devices for agitating animal bedding are constructed and arranged to fit within a cage body. Systems having a cage body and a tumbler positioned within the cage body are disclosed. Methods of testing an animal population for a target pathogen are disclosed. The methods include introducing used animal bedding into a tumbler, introducing a sample collection unit into the tumbler, actuating a closure mechanism to fasten the tumbler, instructing the controller to operate the motor driving the rotating mechanism, retrieving a loaded sample collection unit from the tumbler, and testing the sample for the presence of the target pathogen. Methods of retrofitting a cage body including providing a device for agitating animal bedding are also disclosed.

### CROSS-REFERENCE TO RELATED APPLICATIONS

This application claims priority under 35 U.S.C. § 119(e) to U.S. Provisional Application Ser. No. 63/253,345, titled “ANIMAL BEDDING AGITATOR,” filed on Oct. 7, 2021, which is incorporated herein by reference in its entirety for all purposes.

### FIELD OF TECHNOLOGY

The disclosure relates to devices and methods for agitating animal bedding. In particular, the disclosure relates to devices and methods of collecting samples for testing used animal bedding for pathogens.

### BACKGROUND

Modern laboratory research often requires the use of animal models, such as mice or rats. Sometimes, a facility may maintain up to tens of thousands of these animals at any given time. Care must be taken when large numbers of animals are housed together, because pathogens that may affect experimental results can spread through the population.

There are many steps that a facility may take to reduce the risk of infection among a population of animal models. Housing animals in separate cages, using air filtration systems, and buying certified pathogen free animals from suppliers all reduce the risk of pathogen spread.

Because the risk of infection among a population of animal models can never be completely eliminated, facilities may be required to periodically test their animals for infection.

Conventional methods of testing for the presence of a pathogen in a population of laboratory animals are time and labor intensive and may require the sacrifice of at least some animals. There is a need for improved devices and methods for testing a large population of animals at the same time.

### SUMMARY

In accordance with one aspect, there is provided a device for agitating animal bedding. The device may comprise a tumbler constructed and arranged to fit within a cage body. The tumbler may have a lateral surface, a first longitudinal end, a second longitudinal end, and one or more openings dimensioned to permit air flow into an interior cavity of the tumbler. The device may comprise a closure mechanism constructed and arranged to fasten the tumbler in a closed configuration. The device may comprise a rotating mechanism constructed and arranged to rotate the tumbler. The device may comprise a motor operably connected to the rotating mechanism and configured to drive the rotating mechanism to rotate the tumbler. The device may comprise a controller operably connected to the motor and programmed to operate the motor.

In some embodiments, the device further comprises a gearbox operably connected to the motor and the rotating mechanism. The gear box may be configured to translate a drive force from the motor to the rotating mechanism.

In some embodiments, the rotating mechanism comprises one or more rollers configured to translate a rotational force to the tumbler.

In some embodiments, the tumbler comprises one or more deflectors on an interior side of the lateral surface and/or first and second longitudinal end.

The deflectors may be selected from corrugations, baffles, and combinations thereof.

In some embodiments, the deflectors comprise corrugations having a semi-circular cross-sectional area projecting from the interior side of the lateral surface.

In some embodiments, the deflectors comprise helical baffles on the interior side of the first and second longitudinal ends having a rotation consistent with a direction of rotation of the tumbler.

In some embodiments, the tumbler may further comprise an access port dimensioned to permit passage of the animal bedding. The closure mechanism may comprise at least one fastener configured to fasten the access port.

In some embodiments, the lateral surface of the tumbler comprises a first portion and a second portion. The closure mechanism may be configured to fasten the first portion to the second portion.

In some embodiments, the tumbler is formed of one or more autoclavable materials.

The tumbler may be formed of polypropylene, polycarbonate, or other high temperature plastic or polymer, aluminum, brass, or combinations thereof.

In some embodiments, the controller is programmed to operate the motor for a predetermined amount of time between about 1 minute and 2 hours.

The controller may be programmed to operate the motor intermittently.

In some embodiments, the motor may be configured to drive the rotating mechanism to rotate the tumbler at a rate of between about 0.1 to 60 revolutions per minute (rpm).

In some embodiments, the tumbler comprises a coupler configured to position a sample collection unit in the interior cavity of the tumbler.

In accordance with another aspect, there is provided a system for agitating animal bedding. The system may comprise a cage body. The system may comprise a tumbler positioned within the cage body having a lateral surface, a first longitudinal end, a second longitudinal end, and one or more openings dimensioned to permit air flow into an interior cavity of the tumbler. The system may comprise a closure mechanism constructed and arranged to fasten the tumbler in a closed configuration. The system may comprise a rotating mechanism constructed and arranged to rotate the tumbler. The system may comprise a motor operably connected to the rotating mechanism and configured to drive the rotating mechanism to rotate the tumbler. The system may comprise a controller operably connected to the motor and programmed to operate the motor.

In some embodiments, the tumbler may be reversibly fastened to the cage body.

In some embodiments, the cage body may be connectable to an animal housing environment.

In some embodiments, the closure mechanism and the rotating mechanism may be positioned within the cage body.

In some embodiments, the system further comprises a power source operably connectable to the controller.

In accordance with another aspect, there is provided a method of testing an animal population for at least one target pathogen with a device comprising a tumbler having a lateral surface, a first longitudinal end, a second longitudinal end, and one or more openings dimensioned to permit air flow into an interior cavity of the tumbler, a rotating mechanism constructed and arranged to rotate the tumbler, a motor operably connected to the rotating mechanism and configured to drive the rotating mechanism to rotate the tumbler, and a controller operably connected to the motor and programmed to operate the motor.

The method may comprise introducing used animal bedding from at least one animal cage body into the interior cavity of the tumbler. The method may comprise introducing a sample collection unit into the interior cavity of the tumbler in contact with the used animal bedding. The method may comprise actuating a closure mechanism constructed and arranged to fasten the tumbler in a closed configuration. The method may comprise instructing the controller to operate the motor driving the rotating mechanism for a period of time effective to load the sample collection unit with a sample from the used animal bedding. The method may comprise retrieving the loaded sample collection unit from the interior cavity of the tumbler. The method may comprise testing the sample for presence of the at least one target pathogen.

In some embodiments, the method may comprise introducing used animal bedding from more than one animal cage body into the interior cavity of the tumbler.

In some embodiments, the method may comprise testing the used animal bedding from each animal cage body separately, responsive to the at least one target pathogen being detected in the sample.

In some embodiments, the sample collection unit is a filter paper. Testing the sample may comprise performing a polymerase chain reaction (PCR) test on the sample.

In accordance with another aspect, there is provided a method of retrofitting a cage body. The method may comprise providing a device for agitating animal bedding comprising a tumbler having a lateral surface, a first longitudinal end, a second longitudinal end, and one or more openings dimensioned to permit air flow into an interior cavity of the tumbler and substantially block passage of the animal bedding; a closure mechanism constructed and arranged to fasten the tumbler in a closed configuration; a rotating mechanism constructed and arranged to rotate the tumbler; a motor operably connected to the rotating mechanism and configured to drive the rotating mechanism to rotate the tumbler; and a controller operably connected to the motor and programmed to operate the motor.

The method may further comprise providing a sample collection unit configured to be positioned within the interior cavity of the tumbler.

In some embodiments, the tumbler may be configured to be reversibly fastened to the cage body.

The method may further comprise providing a power source operably connectable to the controller.

The disclosure contemplates all combinations of any one or more of the foregoing aspects and/or embodiments, as well as combinations with any one or more of the embodiments set forth in the detailed description and any examples.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a front perspective view of an animal bedding agitator, according to one embodiment;

FIG. 2 is an exploded view of a tumbler, according to one embodiment;

FIG. 3 is a photograph of an animal bedding agitator inside of an animal cage, according to one embodiment;

FIG. 4 is a flowchart of a method for agitating animal bedding, according to one embodiment; and

FIG. 5 is a photograph of a portion of an animal bedding agitator, according to one embodiment.

### DETAILED DESCRIPTION

Many pathogens of concern to researchers can survive and propagate through soiled cage bedding. Animals used in laboratory research may need to be tested to be pathogen free. The pathogens may be detectable in used animal bedding containing the animal's waste. According to certain testing methods, samples from the used bedding may be tested for infection using laboratory procedures, such as polymerase chain reaction (PCR) testing for genetic markers known to be associated with a particular pathogen of interest. If the pathogen is present, any animals in the contaminated cages must be excluded from the experiments and replaced with new animals. In certain cases, the full colony of the contaminated cages may need to be excluded.

It is possible to test an animal cage bedding for pathogens directly. According to certain methods, a filter paper may be placed into contact with the used animal bedding. Air drawn across the filter paper may be used to collect particulate from the cage bedding onto the filter paper for testing. For instance, the cage bedding may be agitated in the presence of air flow to expose particulate matter to the filter paper that can be tested for the presence of pathogens. However, testing each cage separately increases the number of samples that must be taken, reagents used, and overall cost to keep the animal population pathogen free.

The cost of testing may be lowered by combining the bedding of several animals to be tested at once. According to some methods, used bedding of several animal cages may be collected and tested together. The filter paper may be put into contact with the combined used animal bedding. The filter paper may be agitated and tested for the presence of pathogens. A need exists for a device and method to agitate used bedding from a population of laboratory animals to facilitate pathogen testing.

This disclosure is directed to animal bedding agitators, animal cages containing animal bedding agitators, and methods of using the same. The disclosed devices and methods may provide improved animal bedding agitation without the need to expose an animal to perform the agitation.

In accordance with certain embodiments, an animal bedding agitator, also referred to a “device” herein, is disclosed comprising a tumbler. The tumbler may be constructed and arranged to fit within an animal cage body. For instance, the tumbler may be constructed and arranged to fit within a standard size animal cage body. The animal cage body may be connectable to an animal housing environment. Exemplary animal cage bodies may be sized to house mice, hamsters, gerbils, guinea pigs, rats, chinchillas, or other rodents. Other exemplary animal cage bodies may be sized to house larger mammals, such as rabbits, hares, or ferrets.

The tumbler may have a lateral surface, a first longitudinal end, and a second longitudinal end. The tumbler may include one or more openings dimensioned to permit air flow into the interior cavity of the tumbler. In some embodiments, the first longitudinal end and second longitudinal end may each include one or more openings to permit air flow into the tumbler. In some embodiments, the lateral surface may include one or more openings to permit air flow into the tumbler.

In some embodiments, the one or more openings may be dimensioned to substantially block passage of the animal bedding. Animal bedding is a material, usually organic, placed in animal housing to provide comfort, reduce heat loss, and reduce contamination by waste. Exemplary bedding material may comprise different one or more substrates such as wood, paper, cellulose, cotton, corn, or others. In some embodiments, the one or more openings may be smaller than an average bulk size of the bedding material. In some embodiments, even if the openings are larger than an average bulk size of the bedding material, the device may comprise a baffle, guard, or mesh blocking passage of the average bulk bedding material through the openings. In use, some bedding material may find passage through the openings, as long as a substantial amount of the bedding material remains within the tumbler.

The tumbler may have an open configuration and a closed configuration. In the open configuration, animal bedding may be placed within the tumbler and/or removed from the tumbler. In the closed configuration, animal bedding may generally remain within the tumbler. The device may comprise a closure mechanism constructed and arranged to fasten the tumbler in the closed configuration. In some embodiments, the closure mechanism may comprise a fastener, such as a clasp, clip, claw, or other fastener. In some embodiments, the closure mechanism may comprise a screw thread or a snap closure. In some embodiments, the closure mechanism may comprise a band under tension, such as a rubber band.

In some embodiments, the tumbler may be set to the open configuration by opening an access port. The tumbler may then be set to the closed configuration by closing the access port. Thus, the tumbler may comprise an access port dimensioned to permit passage of the animal bedding. The closure mechanism may comprise at least one fastener configured to fasten the access port.

In some embodiments, the tumbler may be set to the open configuration by separating a first portion and a second portion of the tumbler. The tumbler may be set to the closed configuration by joining the first and second portions. The lateral surface of the tumbler may comprise the first portion and the second portion. The first longitudinal end and second longitudinal end may also comprise a first portion and a second portion, for example, each fixed to the respective portion of the lateral surface. In some embodiments, one end longitudinal end may correspond to one lateral surface portion. The second longitudinal end may correspond to the second lateral surface portion. These embodiments may be combined to form the tumbler that can be opened by separating two portions. In such embodiments, the closure mechanism may be configured to fasten the first portion to the second portion.

The tumbler may be formed of one or more autoclavable materials, e.g., materials known to be safe to be cleaned in an autoclave. In some embodiments, the tumbler may be formed of polypropylene, polypropylene, polycarbonate, or other high temperature plastic or polymer, aluminum, brass, or combinations thereof.

The device may comprise a rotating mechanism constructed and arranged to rotate the tumbler. In some embodiments, the rotating mechanism may be dimensioned to fit within the cage body with the tumbler. In other embodiments, the rotating mechanism may be partially or fully positioned exterior to the cage body. In some embodiments, the rotating mechanism may be formed of an autoclavable material, as previously described. In other embodiments, the rotating mechanism may be sterilized by topical application of a disinfectant.

The rotating mechanism may comprise one or more rollers configured to translate a rotational force to the tumbler. The rollers may be constructed of a rigid rod sufficient to support the weight of the tumbler under maximum expected load. The rollers may be constructed of materials such as brass, aluminum, stainless steel, or other rigid materials, such as rigid polymer materials, for supporting the weight of the tumbler in motion. Multiple rollers may be linked by a belt, chain, or drive shaft configured to coordinate rotation of the rollers.

The rotating mechanism may comprise one or more gears configured to rotate the tumbler. The gears may optionally drive one or more belt, chain, or drive shaft. The gear, chain, belt, and/or drive shaft may translate rotational force to the tumbler. For example, the gear, chain, belt, and/or drive shaft may translate rotational force to a corresponding gear, rotation guide, or other element of the tumbler.

The tumbler may have an exterior tread surface, or a portion of the tumbler may have an exterior tread surface. The tread surface may provide traction to the tumbler for rotation. In some embodiments, the tread surface may be configured to mate with a corresponding surface element of the rotating mechanism. The rotating mechanism, e.g., one or more roller, gear, chain, or belt, may provide rotational force to the tumbler by driving the tread surface of the tumbler. The tread surface may comprise a roughened surface, or one or more notches, grooves, or clefts.

The rotating mechanism may have a guide or friction element positioned to provide traction to the tumbler for rotation. In certain embodiments, the guide or friction element may provide traction to a tumbler that does not have an exterior tread surface. In certain embodiments, the guide or friction element may be utilized with a tumbler that comprises an exterior tread surface. The guide or friction element may be, for example, a rubber or other high friction material contact element. In some embodiments, the guide may be configured to mate with a corresponding element of the tumbler, for example, notches, grooves, or clefts. The guide or friction element may additionally position the tumbler on the device, reducing inconsistency of the tumbler concentricity.

The device may comprise a motor operably connected to the rotating mechanism and configured to drive the rotating mechanism to rotate the tumbler. The motor may be a standard DC motor rated at between 1.5 to 24 volts, for example, 3 to 12 volts. The motor may be configured to drive the rotating mechanism to rotate the tumbler at a rate of between about 0.1 to 60 revolutions per minute (rpm), for example, about 0.1-1 rpm, about 1-20 rpm, about 20-40 rpm, or about 40-60 rpm.

In some embodiments, the motor may provide a variable rotation speed. For example, rotation speed may start slow and ramp up after a predetermined period of time. In some embodiments, rotation speed may be reduced toward the end of the tumbling cycle. Rotation speed may be selected based on the amount or weight of used animal bedding in the tumbler. For example, rotation speed may be increased proportionately with a greater amount and/or weight of used animal bedding. Thus, in some embodiments, the motor may be configurable to operate at varying intensities.

The device may comprise a gearbox. The gearbox may be operably connected to the motor and the rotating mechanism. The gearbox may be configured to translate drive force from the motor to the rotating mechanism. In particular, the gearbox may provide torque conversion from the motor to the rotating mechanism. In some embodiments, the gearbox may increase torque to the rotating mechanism. In some embodiments, the gearbox may decrease torque to the rotating mechanism.

The gearbox may be incorporated in the motor, for example, a gearmotor. Gearmotors are electric motors designed with an integral gearbox. A gearmotor may reduce the number of components and connections in the device. In other embodiments, the gearbox may be external to the motor. The external gearbox may be easier to maintain and troubleshoot, for example, by eliminating the need to take apart the motor for maintenance of the gearbox.

The device may comprise a controller operably connected to the motor and programmed to operate the motor. The controller may be programmed to operate the motor for a predetermined amount of time. In some embodiments, the controller may be programmed to operate the motor for a predetermined amount of time between about 1 minute and 2 hours, for example, between about 1 minute and 3 minutes, between about 1 minute and 10 minutes, between about 10 minutes and 30 minutes, between about 30 minutes and 1 hour, or between about 1 hour and 2 hours. The predetermined amount of time may be selected based on the amount or weight of used animal bedding in the tumbler. In some embodiments, the predetermined amount of time may be increased proportionately with a greater amount and/or weight of used animal bedding. In other embodiments, the controller may be programmed to operate the motor intermittently, for example, over a longer period of time. In other embodiments, the controller maybe programmed to operate the motor continuously, for example, responsive to manual termination.

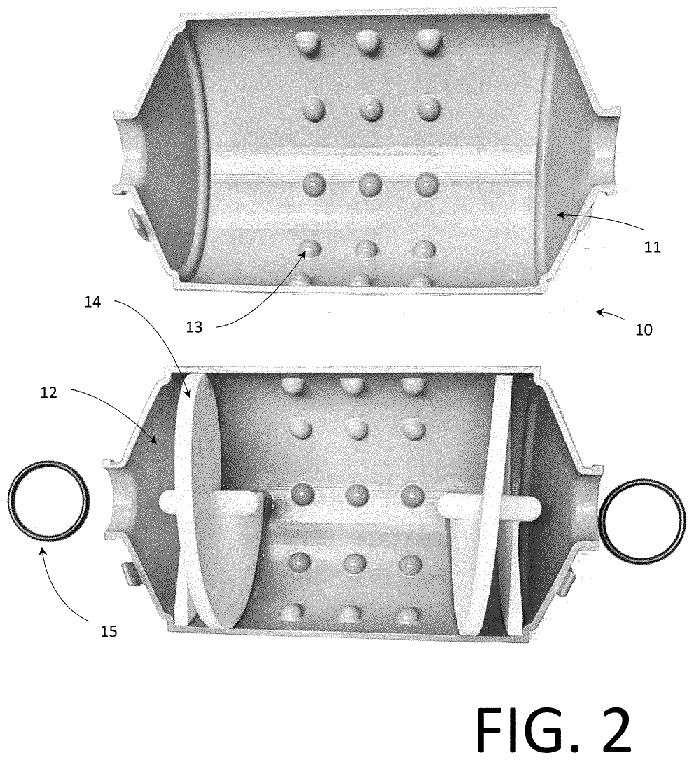

The animal bedding agitator may comprise additional features. In some embodiments, the tumbler comprises one or more deflectors on an interior side of the lateral surface, first longitudinal end, and/or second longitudinal end. The deflectors may comprise corrugations and/or baffles. Exemplary deflectors are shown in FIG. 2.

In some embodiments, the tumbler may include corrugations on an interior side of the lateral surface. The corrugations may contribute to mixing the animal bedding as the tumbler rotates. The corrugations may be dimensioned and positioned to disrupt clumps of bedding during operation of the tumbler, so that the material on the inside of the tumbler mixes more thoroughly. In some embodiments, the corrugations may extend from the interior side of the lateral surface and project towards the interior of the tumbler. In some embodiments, the corrugations may be formed of indentations on the interior side of the lateral surface. In some embodiments, the corrugations may comprise projections and indentations. The corrugations may have any cross-sectional area, for example, semi-circular, rectangular, triangular, or others. The corrugations may be molded from the tumbler surface or coupled to the tumbler surface. The corrugations may be distributed on the lateral surface in a regular pattern or randomized.

The tumbler may additionally or alternatively include baffles on an interior side of either or both longitudinal ends. The baffles may be dimensioned and positioned to redirect animal bedding toward the center of the interior cavity and away from the openings. The baffles may be dimensioned and positioned to reduce blockage of air flow by the animal bedding. The baffles may be dimensioned and positioned to direct air flow within the tumbler. In some embodiments, the baffles may be dimensioned and positioned to control turbulence and/or pressure drop within the tumbler during operation. Baffle spacing and angular arrangement may be selected to control any of the disclosed properties. The baffles may be straight, angled, helical, disk and doughnut, comprise concentric circles, rectangles, or triangles, be formed of combinations thereof, or others. The baffles may be molded from the tumbler surface or coupled to the tumbler surface.

The animal bedding agitator may comprise a coupler configured to position the sample collection unit in the interior cavity of the tumbler. The coupler may be positioned on the interior side of the lateral surface, first longitudinal end, or second longitudinal end. The coupler may position the sample collection unit while allowing the sample collection unit to contact the animal bedding as the tumbler rotates. The coupler may comprise a clip, clasp, or other fastener. The coupler may comprise a pocket or slot having a window to allow contact with the animal bedding.

In accordance with another aspect, there is provided a system for agitating animal bedding. The system may comprise a cage body. The system may comprise a tumbler positioned within the cage body. The system may comprise a closure mechanism, a rotating mechanism, a motor, and a controller, as previously described. In some embodiments, one or more of the closure mechanism, the rotating mechanism, the motor, and the controller are positioned within the animal cage body with the tumbler. In other embodiments, one or more of the closure mechanism, the rotating mechanism, the motor, and the controller are positioned partially or wholly outside the cage body.

In some embodiments, the components positioned within the cage body are reversibly fastened to the cage body. For example, the components positioned within the cage body may snap or clip onto a coupler within the cage body or a rim of the cage body, to reduce motion of the device within the cage body while in use. Any component fastened to the cage body may be easily removable.

The system may comprise a power source connectable to the controller and/or to the motor. The power source may be an electrical outlet. The power source may be a battery source supplying between 1.5 and 24 volts, for example, 3 and 12 volts of power. The power source may comprise two D-cell batteries connected in series. The power source may comprise a lithium ion battery. In some embodiments, the battery source may be rechargeable. In other embodiments, the battery source may be a single use battery.

This disclosure also provides methods of testing used animal bedding for one or more target pathogens. Exemplary target pathogens that may be tested using the methods disclosed herein include viral pathogens, bacterial pathogens, fungal pathogens, or any target pathogen detectable from an animal bedding sample, e.g., an animal bedding sample containing animal waste, bodily fluids, fur, and/or dander.

The methods may be performed in the device for agitating animal bedding disclosed herein. For instance, the methods may be performed in the system for agitating animal bedding disclosed herein. Thus, in certain embodiments, the methods of this disclosure may be performed in an animal cage body. The animal cage body may be configured to be connectable to an animal housing environment. For example, the animal cage body may comprise mating elements configured to connect to the animal housing environment. Optionally, the animal cage body may be positioned in the animal housing environment. The animal housing environment may provide environmental controls to the animal cage body, such as exhaust air filtration. For instance, the animal housing environment may comprise an exhaust air filtration unit connectable to the cage body. In other embodiments, the methods of the disclosure may be performed in a container having an exhaust air filtration system to draw air through the tumbler.

By keeping the tumbler and, optionally, one or more additional components of the animal bedding agitator in an animal cage body, used animal bedding can be sampled and agitated in the same area of the laboratory where the sampled animals are maintained, limiting the risk of cross contamination. Additionally, animal cages for research institutions typically include environmental controls, and specifically exhaust air filtration. The air flow provided by the exhaust air filtration of an animal caging system may therefore provide the air flow to ensure that particulate matter is likely to be deposited on the sample collection unit.

The methods may comprise introducing used animal bedding from at least one animal cage body into the interior cavity of the tumbler with a sample collection unit to contact the used animal bedding. The sample collection unit may be a filter paper, adhesive paper, swab, or any other element capable of collecting a biological sample. The sample collection unit may be placed in a coupler or otherwise coupled to a surface of the tumbler. In other embodiments, the sample collection unit may be loose within the tumbler with the animal bedding. The methods may comprise actuating the closure mechanism of the tumbler to fasten the tumbler in the closed configuration with the used animal bedding and sample collection unit inside.

The methods may comprise instructing the controller to operate the motor driving the rotating mechanism for a period of time effective to load the sample collection unit with a sample from the used animal bedding. In some embodiments, the controller may be programmed to drive the rotating mechanism for the time effective to load the sample collection unit. In other embodiments, the methods may comprise instructing the controller to begin operation of the motor and subsequently instructing the controller to cease operation of the motor. The controller may be operated directly or through an external control unit comprising, for example, a computer or mobile device programmed to interface with the controller. The controller may be operably connected to the external control unit by direct or wireless network connection.

The methods may comprise retrieving the loaded sample collection unit from the interior cavity of the tumbler. Thus, the methods may comprise actuating the closure mechanism of the tumbler to position the tumbler in the open configuration. The loaded sample collection unit may be retrieved from the coupler. In other embodiments, the methods may comprise separating the loaded sample collection unit from the used animal bedding within the tumbler.

The methods may comprise testing the loaded sample collection unit for presence of the at least one target pathogen. The methods may comprise separating the sample from the loaded sample collection unit and performing one or more laboratory tests on the sample to determine whether the target pathogen is detected or not detected in the sample. Exemplary laboratory tests include antigen tests, polymerase chain reaction (PCR) tests, and others.

In some embodiments, the method may comprise introducing used animal bedding from more than one animal cage body. With such a method, a large number of animal cage bodies may be tested more efficiently. If the target pathogen is not detected in the animal cage bedding from the animal cage bodies, it can be determined that the target pathogen has not contaminated any of the tested animal cage bodies. However, if the target pathogen is detected, it is unknown which animal cage bodies are contaminated with the target pathogen. Thus, in some embodiments, the methods may further comprise testing used animal bedding from each animal cage body separately, responsive to the target pathogen being detected in the sample.

In accordance with certain exemplary embodiments of the method of testing animal bedding for a target pathogen disclosed herein, a first portion of the tumbler may be charged with used animal bedding. The first portion of the tumbler may be charged with the sample collection unit. The first portion of the tumbler may be covered with a second portion of the tumbler and closed using the closure mechanism. The controller may be activated to operate the motor. For instance, the controller may supply a voltage to the motor for a predetermined amount of time.

After the operation time has elapsed, the tumbler may be disassembled to obtain the sample collection unit. The sample collection unit may be a filter paper. PCR analysis may be performed on the sample, for example, as collected on the filter paper, to detect the presence of pathogens that may require animals to be excluded from an experimental protocol.

In accordance with another aspect, there is provided a method of retrofitting a cage body. The method may comprise providing one or more of the components for agitating animal bedding described herein. For instance, the methods may comprise providing a device for agitating animal bedding. The methods may comprise providing one or more of the tumbler, closure mechanism, rotating mechanism, a motor, and controller. The methods may comprise providing the power source or a connection to the power source. The methods may comprise providing a sample collection unit.

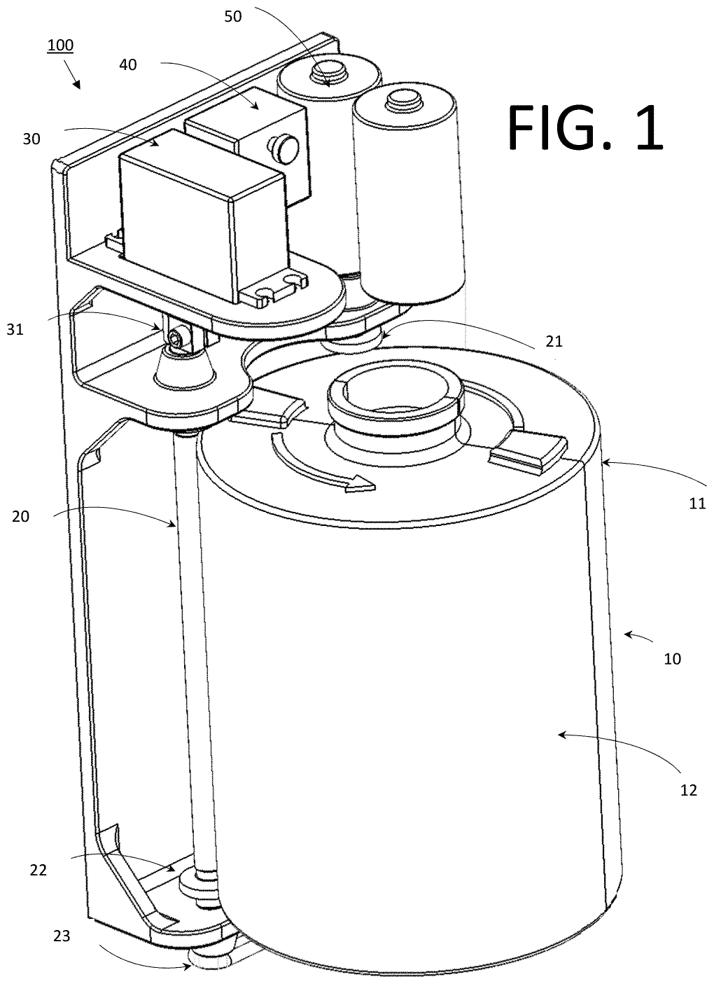

Referring specifically to the exemplary embodiment of FIG. 1, an animal bedding agitator 100 may include a tumbler 10 having a first portion 11 and a second portion 12. The first portion 11 and second portion 12 of tumbler 10 may be separable to allow a user access to the interior cavity of the tumbler 10. When assembled, tumbler 10 of FIG. 1 may form a cylinder that is tapered towards its lateral end points. However, the tumbler may have any cross-sectional area. The lateral end points of tumbler 10 are each configured to have an opening, to allow air to circulate into the interior of tumbler 10. The entirety of tumbler 10 may be removable from animal bedding agitator 100 for the purposes of charging, discharging and cleaning, to avoid obstructions or cross contamination with other components.

Tumbler 10 may be sized to accommodate any amount of used animal bedding. According to one embodiment, tumbler 10 is sized to accommodate approximately 450 ml of animal bedding, although it is expected that a lower volume of bedding would be used in ordinary operation. For example, pathogens may be detectable from a sample of as little as 15 ml of material tumbled with filter paper. Thus, in accordance with certain embodiments, tumbler 10 may be sized to accommodate about 15 ml to about 100 ml, about 100 ml to about 200 ml, or about 200 ml to about 450 ml of used animal bedding.

In some embodiments, multiple cages may be sampled and tumbled in animal bedding agitator 100 simultaneously. However, because testing can only detect the presence or absence of pathogens in the combined sample, testing too many samples at once risks excluding healthy animals from an experimental protocol where a pathogen may only be present in a few or one of the animals whose bedding is being tested. Thus, in accordance with certain embodiments, each cage, or smaller groups of cages, may be tested separately following a positive detection of a target pathogen.

Animal bedding agitator 100 comprises two rollers as a rotation mechanism. Thus, when configured for operation, tumbler 10 may rest on a first roller 20, and a second roller 21. Each first roller 20 and second roller 21 include roller guides 22 for maintaining the position and alignment of tumbler 10 on first roller 20 and second roller 21, and for providing additional friction to translate the rotation of first roller 20 and second roller 21 to tumbler 10. According to one embodiment, roller guides 22 are positioned to contact tumbler 10 on a tread surface comprising a notch, groove, or cleft in tumbler 10. The position of roller guides 22 along the length of rod 20 may be adjustable, so that roller guides 22 may be repositioned by a user to correct any errors in the alignment of tumbler 10 on rollers 20, 21. In other embodiments, the use of a notch, groove or cleft in tumbler 10 may not be necessary for alignment.

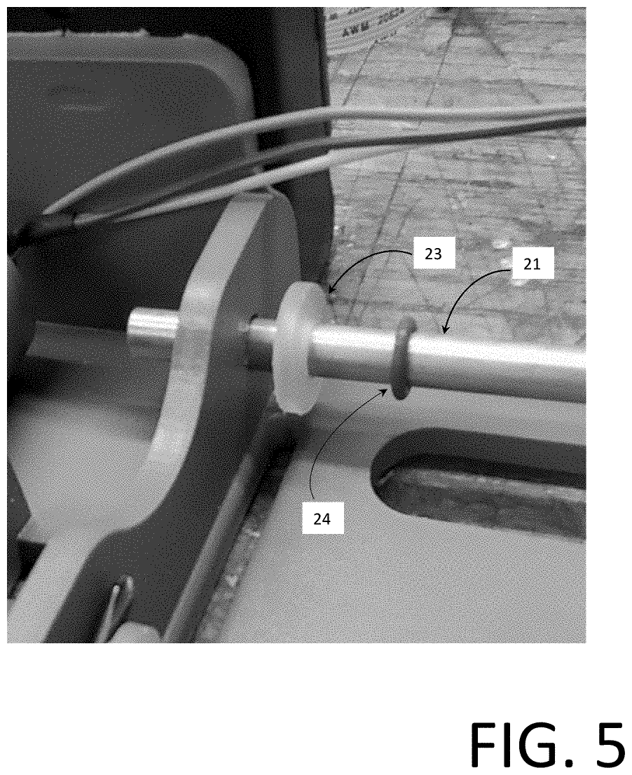

According to one embodiment, a rubber ring 24 (shown in FIG. 5), is positioned around each roller 20, 21 to contact tumbler 10 and provide friction. The position of rubber ring 24 along the length of rollers 20, 21 may be adjustable.

According to one embodiment, first roller 20 and second roller 21 are positioned parallel to one another, separated by a distance roughly equivalent to half the radius of tumbler 10. The separation of the rollers may be varied according to the needs of the application without departing from the spirit of the disclosure. The first roller 20 and second roller 21 are connected to one another by a roller linkage 23, configured to translate the rotation of one roller to the second roller. Roller linkage 23 may be in the form of a belt, chain, or drive shaft. According to one embodiment, roller linkage 23 comprises a pully positioned at the distal longitudinal ends of first roller 20 and second roller 21 connected by a belt.

Exemplary animal bedding agitator 100 includes a motor 30 configured to drive first roller 21. Motor 30 is connected to first roller 20 through a motor linkage 31. When powered, motor 30 causes a rotational force that is translated through motor linkage 31 to first roller 20, and through roller linkage 23 to second roller 21. In certain exemplary embodiments, a low rate of rotation may be induced to provide the necessary agitation the amount of agitation necessary to cover filter paper is relatively small. According to one embodiment, motor 30 is configured to rotate tumbler 10 at a rate of approximately 4 rotations per minute. However, the rate of rotation may be varied without departing from the spirit of this disclosure and may include rates of between 0.1 rotation per minute and 60 rotations per minute, as previously described. Exemplary motor 30 is powered by a battery 40.

Animal bedding agitator 100 comprises a controller. The exemplary controller shown in FIG. 1 is timer 50. Timer 50 may be a conventional electronic timer configured to supply voltage from battery 40 to motor 30 for a set amount of time corresponding to a particular research protocol. According to one embodiment, timer 50 is configured to supply voltage for 10 minutes once activated. According to another embodiment, timer 50 is configured to supply voltage for 1 hour.

According to another embodiment, timer 50 may be a programmable timer capable of a period of delay before supplying voltage to motor 30. Timer 50 may be a programmable timer capable of supplying voltage intermittently, according to a pre-set schedule. In one example, timer 50 may be configured to supply voltage to motor 30 once per day for 10 minutes a day. In another example, timer 50 may be configured to supply voltage to motor 30 one hour per day, one day a week. Other intermittent programs are within the scope of the disclosure.

Referring to the exemplary embodiment of FIG. 2, the interior of tumbler 10 may include corrugations 13 configured to disrupt clumps of bedding during operation, so that the material on the inside of the tumbler mixes more thoroughly. In one embodiment, the corrugations 13 are in the form of semi-spherical bumps or ridges projecting towards the interior of tumbler 10. In another embodiment, corrugations 13 are in the form grooves or depressions in the interior surface of tumbler 10. Corrugations 13 may be distributed in a regular pattern, or they may be randomized.

The interior of tumbler 10 may also include baffles 14 that are designed to maintain an open airway between the interior and exterior of tumbler 40, while redirecting material inside the tumbler away from the openings. In one embodiment, the baffles 14 are in the form of a screws having a rotation that is consistent with the direction of rotation of tumbler 10, so that material that is adjacent to the screws is always directed back towards the interior of tumbler 10. Baffles 14 may also be in the form of slats, a screen, or other forms that maintain open air flow and prevent material from falling out of tumbler 10 during operation.

Tumbler 10 may also include a closure mechanism 15 designed to keep the first half 11 and second half 12 of tumbler 10 in contact with one another during operation. In some embodiments, closure mechanism 15 is in the form of rubber bands. According to other embodiments, closure mechanism 15 may be in the form of a clasp, clip, or registration points that interlock via friction, or similar mechanisms of keeping first portion 11 and second portion 12 of tumbler 10 connected.

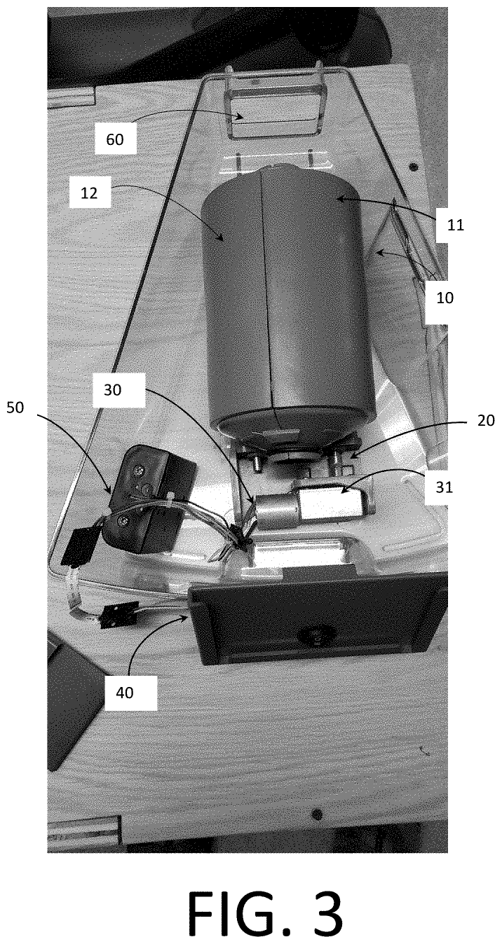

Referring to the exemplary embodiment of FIG. 3, animal bedding agitator 100 may be sized and proportioned to fit within the interior of an animal cage 60. In the exemplary embodiment of FIG. 3, rotating mechanism 20, motor 30, motor linkage 31, and controller 50 are positioned within animal cage 60, while power source 40 is positioned outside animal cage 60. However, in some embodiments, one or more of these elements may be positioned within animal cage 60 or partially or wholly outside animal cage 60.

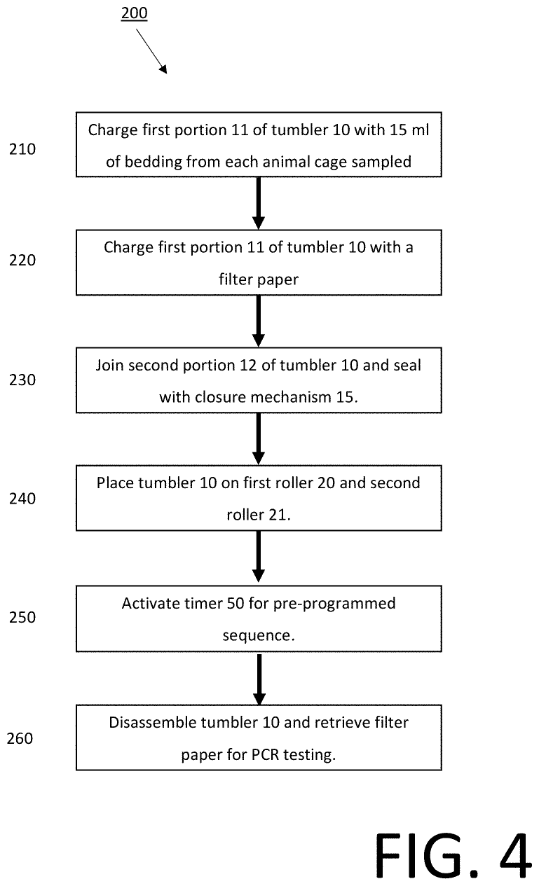

Referring to the exemplary embodiment of FIG. 4, a method of testing the used bedding of laboratory animals 200 using an animal bedding agitator 100 is described. In step 210, first portion 11 of tumbler 10 is charged with approximately 15 ml of animal bedding from each of 10 animal cages. In step 220, a unit of filter paper is added to the first portion 11 of tumbler 10. In step 230, second portion 12 of tumbler 10 is joined to first portion 11, and secured using the closure mechanism 15. In step 240, tumbler 10 is placed on first roller 20 and second roller 21. In step 250, timer 40 is activated, and supplies voltage to motor 30 for a pre-programmed amount of time, ten minutes in this example. In step 260, once the pre-programmed amount of time has elapsed, tumbler 10 is disassembled, and the filter paper is removed for further testing, PCR in this embodiment.

Referring to the exemplary embodiment of FIG. 5, animal bedding agitator 100 may include a roller guide 22 and/or rubber ring 24 shown positioned on roller 21. However, the roller guide 22 and/or rubber ring 24 may be positioned on one or both rollers 20, 21.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Any references to front and back, left and right, top and bottom, or upper and lower and the like are intended for convenience of description, not to limit the present systems and methods or their components to any one positional or spatial orientation.

Any references to embodiments or elements herein referred to in the singular may also embrace embodiments including a plurality of these elements, and any references in plural to any embodiment or element herein may also embrace embodiments including only a single element. References in the singular or plural form are not intended to limit the presently disclosed components or elements to single or plural configurations.

What is claimed is:

1. A device for agitating animal bedding, comprising:
a tumbler constructed and arranged to fit within a cage body, the tumbler having a lateral surface, a first longitudinal end, a second longitudinal end, and one or more openings dimensioned to permit air flow into an interior cavity of the tumbler;
a closure mechanism constructed and arranged to fasten the tumbler in a closed configuration;
a rotating mechanism constructed and arranged to rotate the tumbler;
a motor operably connected to the rotating mechanism and configured to drive the rotating mechanism to rotate the tumbler; and
a controller operably connected to the motor and programmed to operate the motor.

2. The device of claim 1, further comprising a gearbox operably connected to the motor and the rotating mechanism, configured to translate a drive force from the motor to the rotating mechanism.

3. The device of claim 1, wherein the rotating mechanism comprises one or more rollers configured to translate a rotational force to the tumbler.

4. The device of claim 1, wherein the tumbler comprises one or more deflectors on an interior side of the lateral surface and/or first and second longitudinal ends.

5. The device of claim 4, wherein the deflectors are selected from corrugations, baffles, and combinations thereof.

6. The device of claim 5, wherein the deflectors comprise corrugations having a semi-circular cross-sectional area projecting from the interior side of the lateral surface.

7. The device of claim 5, wherein the deflectors comprise helical baffles on the interior side of the first and second longitudinal ends having a rotation consistent with a direction of rotation of the tumbler.

8. The device of claim 1, wherein the tumbler is formed of one or more autoclavable materials.

9. The device of claim 8, wherein the tumbler is formed of polypropylene, polypropylene, polycarbonate, or other high temperature plastic or polymer, aluminum, brass, or combinations thereof.

10. The device of claim 1, wherein the controller is programmed to operate the motor for a predetermined amount of time between about 1 minute and 2 hours.

11. The device of claim 1, wherein the controller is programmed to operate the motor intermittently.

12. The device of claim 1, wherein the tumbler comprises a coupler configured to position a sample collection unit in the interior cavity of the tumbler.

13. A system for agitating animal bedding comprising:
a cage body;
a tumbler positioned within the cage body having a lateral surface, a first longitudinal end, a second longitudinal end, and one or more openings dimensioned to permit air flow into an interior cavity of the tumbler;
a closure mechanism constructed and arranged to fasten the tumbler in a closed configuration;
a rotating mechanism constructed and arranged to rotate the tumbler;
a motor operably connected to the rotating mechanism and configured to drive the rotating mechanism to rotate the tumbler; and
a controller operably connected to the motor and programmed to operate the motor.

14. The system of claim 13, wherein the tumbler is reversibly fastened to the cage body.

15. The system of claim 13, wherein the cage body is connectable to an animal housing environment.

16. The system of claim 13, wherein the closure mechanism and the rotating mechanism are positioned within the cage body.

17. A method of testing an animal population for at least one target pathogen with a device comprising a tumbler having a lateral surface, a first longitudinal end, a second longitudinal end, and one or more openings dimensioned to permit air flow into an interior cavity of the tumbler, a rotating mechanism constructed and arranged to rotate the tumbler, a motor operably connected to the rotating mechanism and configured to drive the rotating mechanism to rotate the tumbler, and a controller operably connected to the motor and programmed to operate the motor, the method comprising:
introducing used animal bedding from at least one animal cage body into the interior cavity of the tumbler;
introducing a sample collection unit into the interior cavity of the tumbler in contact with the used animal bedding;
actuating a closure mechanism constructed and arranged to fasten the tumbler in a closed configuration;
instructing the controller to operate the motor driving the rotating mechanism for a period of time effective to load the sample collection unit with a sample from the used animal bedding;
retrieving the loaded sample collection unit from the interior cavity of the tumbler; and
testing the sample for presence of the at least one target pathogen.

18. The method of claim 17, comprising introducing used animal bedding from more than one animal cage body into the interior cavity of the tumbler.

19. The method of claim 18, further comprising testing the used animal bedding from each animal cage body separately responsive to the at least one target pathogen being detected in the sample.

20. The method of claim 17, wherein the sample collection unit is a filter paper and testing the sample comprises performing a polymerase chain reaction (PCR) test on the sample.

21. A method of retrofitting a cage body comprising providing a device for agitating animal bedding, comprising:
a tumbler having a lateral surface, a first longitudinal end, a second longitudinal end, and one or more openings dimensioned to permit air flow into an interior cavity of the tumbler and substantially block passage of the animal bedding;
a closure mechanism constructed and arranged to fasten the tumbler in a closed configuration;
a rotating mechanism constructed and arranged to rotate the tumbler;
a motor operably connected to the rotating mechanism and configured to drive the rotating mechanism to rotate the tumbler; and
a controller operably connected to the motor and programmed to operate the motor.

22. The method of claim 21, further comprising providing a sample collection unit configured to be positioned within the interior cavity of the tumbler.

23. The method of claim 22, wherein the tumbler is configured to be reversibly fastened to the cage body.
